(19) United States
(12) Patent Application Publication
(10) Pub. No.: US 2022/0304807 A1
(43) Pub. Date: Sep. 29, 2022

(54) PUMP ASSEMBLY FOR AN IMPLANTABLE INFLATABLE DEVICE

(71) Applicant: Boston Scientific Scimed, Inc., Maple Grove, MN (US)

(72) Inventors: Noel Smith, Co. Kilkenny (IE); Keith R. Maile, New Brighton, MN (US); Thomas Andrew Albrecht, Edina, MN (US); Daragh Nolan, Co. Waterford (IE); Brian P. Watschke, Minneapolis, MN (US); Thomas Sinnott, Wexford (IE); Richard Percy, Co. Cork (IE); Bryan Duane Johnson, Mahtomedi, MN (US); Laurence Norris, Waterford (IE); John Gildea, Kildare (IE); Eduardo Marcos Larangeira, Tipperary (IE)

(21) Appl. No.: 17/655,937

(22) Filed: Mar. 22, 2022

Related U.S. Application Data
(60) Provisional application No. 63/200,737, filed on Mar. 25, 2021.

(51) Int. Cl. A61F 2/26; A61F 2/48

(52) U.S. Cl. CPC A61F 2/26; A61F 2/482; A61F 2250/0013

(57) ABSTRACT

An implantable fluid operated device may include a fluid reservoir configured to hold fluid, an inflatable member, and a pump assembly configured to transfer fluid between the fluid reservoir and the inflatable member. The pump assembly may include one or more fluid pumps and one or more valves. One or more sensing devices may be positioned within fluid passageways of the fluid operated device. The electronic control system may control operation of the pump assembly based on fluid pressure measurements and/or fluid flow measurements received from the one or more sensing devices. The pump assembly may include a piezoelectric pump. The one or more sensing devices may include one or more pressure transducers positioned in the fluid passageways, one or more strain gauges measuring deflection of piezoelectric elements, voltage input/output at one or more piezoelectric elements, and other types of sensing devices.

### CROSS-REFERENCE TO RELATED APPLICATION

This application claims priority to U.S. Provisional Patent Application No. 63/200,737, filed on Mar. 25, 2021, entitled “PUMP ASSEMBLY FOR AN IMPLANTABLE INFLATABLE DEVICE”, the disclosure of which is incorporated by reference herein in its entirety.

### TECHNICAL FIELD

This disclosure relates generally to bodily implants, and more specifically to bodily implants including a pump.

### BACKGROUND

Active implantable fluid operated inflatable devices often include one or more pumps that regulate a flow of fluid between different portions of the implantable device to provide for inflation and deflation of one or more fluid fillable implant components of the device. One or more valves can be positioned within fluid passageways of the device to direct and control the flow of fluid so as to achieve inflation, deflation, pressurization, depressurization, activation, deactivation and the like of the different fluid fillable implant components of the device. In some implantable fluid operated inflatable devices, sensors can be used to monitor fluid pressure and/or fluid volume and/or fluid flow rate within fluid passageways of the device. Accurate monitoring of conditions within the device, including pressure monitoring and flow monitoring, may provide for improved control of device operation, improved diagnostics, and improved efficacy of the device.

### SUMMARY

According to an aspect, an implantable fluid operated inflatable device includes a fluid reservoir; an inflatable member; and a pump and valve assembly configured to transfer fluid between the fluid reservoir and the inflatable member. The pump assembly includes a manifold, including a housing; at least one valve and at least one pump positioned in a fluid passageway formed in the housing; a first fluid port in fluidic communication with the fluid reservoir; and a second fluid port in fluidic communication with the inflatable member. The device also includes an electronic control system controlling operation of the pump and valve assembly; and at least one pressure sensing device in communication with the electronic control system.

In some implementations, the at least one valve and the at least one pump includes a first pump and a first valve positioned in a first fluid passageway and in fluidic communication with the first fluid port; and a second pump and a second valve positioned in a second fluid passageway and in fluidic communication with the second fluid port. The at least one pressure sensing device can include a first pressure sensing device positioned in the first fluid passageway and configured to measure a pressure of fluid flowing through the first fluid port and to transmit the measured pressure to the electronic control system; and a second pressure sensing device positioned in the second fluid passageway and configured to measure a pressure of fluid flowing through the second fluid port and to transmit the measured pressure to the electronic control system.

In some implementations, the at least one valve and the at least one pump includes a dual piezoelectric pump manifold configuration, including a first piezoelectric pump; a second piezoelectric pump; and a fluid channel providing for fluidic communication between the first piezoelectric pump and the second piezoelectric pump. The first piezoelectric pump can include a first chamber; a first piezoelectric diaphragm positioned along an edge portion of the first chamber; a first check valve at an inlet end of the first chamber; and a second check valve at an outlet end of the first chamber, the second check valve of the first piezoelectric pump selectively providing fluidic communication between the first chamber and the fluid channel. The second piezoelectric pump can include a second chamber; a second piezoelectric diaphragm positioned along an edge portion of the second chamber; a first check valve at an inlet end of the second chamber, the first check valve of the second piezoelectric pump selectively providing fluidic communication between the fluid channel and the second chamber; and a second check valve at an outlet end of the second chamber. In some implementations, a pumping cycle of the dual piezoelectric pump manifold configuration includes a first phase including a supply stroke of the first piezoelectric diaphragm in coordination with a pressure stroke of the second piezoelectric diaphragm; and a second phase including a pressure stroke of the first piezoelectric diaphragm in coordination with a supply stroke of the second piezoelectric diaphragm. In some implementations, in the first phase, fluid is drawn into the first chamber through the first check valve of the first piezoelectric pump, and fluid is expelled from the second chamber through the second check valve of the second piezoelectric pump; and in the second phase, fluid is expelled from the first chamber and into the fluid channel through the second check valve of the first piezoelectric pump, and fluid is drawn from the fluid channel into the second chamber through the first check valve of the second piezoelectric pump.

In some implementations, the housing of the manifold is made of an injection molded metal material, machined metal material and the like, with the at least one pump and the at least one valve positioned in a sealed fluid passageway defined in the injection molded metal material, such that the manifold is a hermetic manifold.

In some implementations, the pump assembly includes a pump assembly housing, and wherein the manifold and the electronic control system are received in the pump assembly housing. The manifold can be a hermetic manifold, such that components of the electronic control system within the pump assembly housing are isolated from fluid flowing through the hermetic manifold.

In some implementations, the at least one pressure sensing device includes a first pressure sensing device positioned proximate a fluid port of the reservoir; and a second pressure sensing device positioned proximate a fluid port of the inflatable member. The first pressure sensing device can include a first diaphragm positioned in a fluid passageway proximate the reservoir, facing the reservoir; and at least one first strain gauge mounted on the first diaphragm, the at least one first strain gauge being configured to measure a deflection of the first diaphragm and to transmit the measured deflection to the electronic control system. The second pressure sensing device can include a second diaphragm positioned in a fluid passageway proximate the fluid port of the inflatable member, facing the inflatable member; and at least one second strain gauge mounted on the second diaphragm, the at least one second strain gauge being configured to measure a deflection of the second diaphragm and to transmit the measured deflection to the electronic control system.

In some implementations, the at least one sensing device includes at least one piezoelectric element positioned in a fluid passageway of the implantable fluid operated device and configured to sense a fluid pressure level in the fluid passageway based on an input voltage level applied to the piezoelectric element and an output voltage level measured at the piezoelectric element.

In some implementations, the electronic control system includes a printed circuit board including a processor configured to receive pressure level measurements from the at least one sensing device; apply a control algorithm based on the received pressure level measurements; and control operation of the at least one valve and the at least one pump in accordance with the applied control algorithm.

In some implementations, the implantable fluid operated device is an artificial urinary sphincter or an inflatable penile prosthesis.

In another general aspect, an implantable fluid operated inflatable device includes a fluid reservoir; an inflatable member; a pump assembly received in a housing and configured to transfer fluid between the fluid reservoir and the inflatable member, and an electronic control system. The pump assembly can include a manifold; and a pump and valve device received in the manifold. The electronic control system can be configured to control operation of the pump and valve device.

In some implementations, the manifold is a hermetic manifold, and the electronic control system includes a first portion received in an electronics compartment of the housing, isolated from fluid flowing through the manifold. In some implementations, the electronic control system includes a second portion that is external to the implantable fluid operated inflatable device, and is configured to communicate with of the first portion of the electronic control system, wherein the second portion is configured to receive user inputs, and to output information to the user.

In some implementations, the pump and valve device is a dual piezoelectric pump and valve configuration device, including a first piezoelectric pump in fluidic communication with a second piezoelectric pump via a fluid channel in a manifold or housing. The first piezoelectric pump can include a first chamber; a first piezoelectric element and diaphragm positioned along an edge portion of the first chamber; a first check valve at an inlet end of the first chamber; and a second check valve at an outlet end of the first chamber, the second check valve of the first piezoelectric pump selectively providing fluidic communication between the first chamber and the fluid channel. The second piezoelectric pump can include a second chamber; a second piezoelectric element and diaphragm positioned along an edge portion of the second chamber; a first check valve at an inlet end of the second chamber, the first check valve of the second piezoelectric pump selectively providing fluidic communication between the fluid channel and the second chamber; and a second check valve at an outlet end of the second chamber. In some implementations, in an inflation mode, the electronic control system is configured to alternately apply a voltage input to the first piezoelectric element and the second piezoelectric element to cause fluid to flow through the dual piezoelectric pump manifold configuration in a first direction, from the fluid reservoir toward the inflatable member; and in a deflation mode, the electronic control system is configured to alternately apply a voltage input to the first piezoelectric element and the second piezoelectric element to cause fluid to flow through the dual piezoelectric pump manifold configuration in a second direction, from the inflatable member toward the reservoir.

### BRIEF DESCRIPTION OF THE DRAWINGS

FIG. 1 is a block diagram of an implantable fluid operated inflatable device according to an aspect.

FIGS. 2A and 2B illustrate example implantable fluid operated inflatable devices according to an aspect.

FIG. 3 is a schematic diagram of a fluid architecture of a pump assembly of an implantable fluid operated inflatable device according to an aspect.

FIGS. 4A and 4B are perspective views of an example manifold of an example pump assembly according to an aspect.

FIGS. 5A and 5B are perspective views of the example manifold installed in an example pump assembly of an implantable fluid operated inflatable device according to an aspect.

FIGS. 6A-6C schematically illustrate operation of an example piezoelectric pump of an implantable fluid operated inflatable device according to an aspect.

FIGS. 7A-7C schematically illustrate operation of an example dual piezoelectric pump & valve manifold configuration of an implantable fluid operated inflatable device according to an aspect.

FIG. 8 is a block diagram of operation of an example dual piezoelectric pump & valve manifold configuration of an implantable fluid operated inflatable device according to an aspect.

FIGS. 9A and 9B are schematic views of implantable fluid operated inflatable devices including inline pressure sensing devices according to an aspect.

FIGS. 10A-10C are graphs illustrating the effect of changes in atmospheric pressure on measured pressure in an implantable fluid operated inflatable device.

FIGS. 11A-11D are graphs illustrating the effect of an impulse at an inflatable member on measured pressure in an implantable fluid operated inflatable device.

FIGS. 12A-12D are graphs illustrating the effect of an impulse at a reservoir on measured pressure in an implantable fluid operated inflatable device.

FIGS. 13A-13D are graphs illustrating the effect of a component failure or blockage in a fluid passageway on measured pressure in an implantable fluid operated inflatable device.

FIG. 14A is a top view, and FIGS. 14B and 14C are side views, of an example diaphragm fitted with strain gauges for measurement of deflection of the diaphragm.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

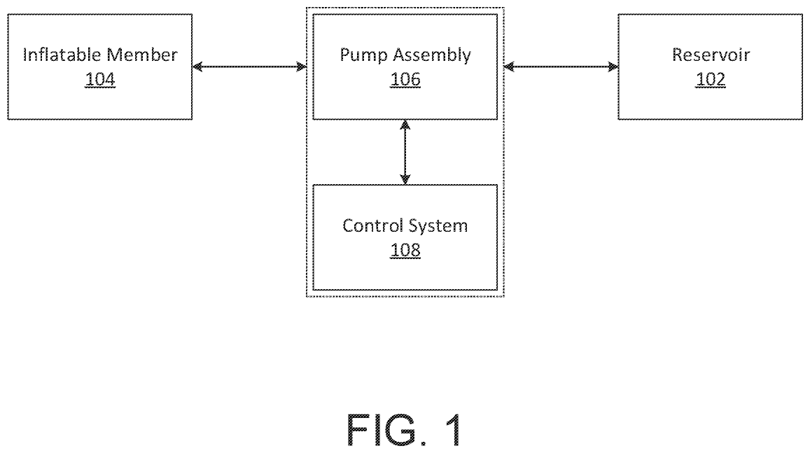

FIG. 1 is a block diagram of an example implantable fluid operated inflatable device 100. The example device 100 shown in FIG. 1 includes a fluid reservoir 102, an inflatable member 104, and a pump assembly 106 configured to transfer fluid between the fluid reservoir 102 and the inflatable member 104. In some implementations, the example device 100 includes a control system 108. In some implementations, the control system 108 is an electronic control system 108. The control system 108 may provide for the monitoring and/or control of the operation of various components of the pump assembly 106 and/or communication with one or more sensing device(s) within the implantable fluid operated inflatable device 100 and/or communication with one or more external device(s). The fluid reservoir 102, the inflatable member 104, and the pump assembly 106 may be internally implanted into the body of the patient. In some implementations, the control system 108 is coupled to or incorporated into the pump assembly 106. In some implementations, at least a portion of the control system 108 is separate or spaced from the pump assembly 106. In some implementations, some modules of the control system 108 are coupled to or incorporated into the pump assembly 106, and some modules of the control system 108 are separate from the pump assembly 106. For example, in some implementations, some modules of the control system 108 are included in an external device that is in communication other modules of the control system 108 included within the implanted device 100. In some implementations, the pump assembly 106 is electronically controlled. In some implementations, the pump assembly 106 is manually controlled.

In some examples, electronic monitoring and control of the fluid operated device 100 may provide for improved patient control of the device, improved patient comfort, and improved patient safety. In some examples, electronic monitoring and control of the fluid operated device 100 may afford the opportunity for tailoring of the operation of the device 100 by the physician without further surgical intervention.

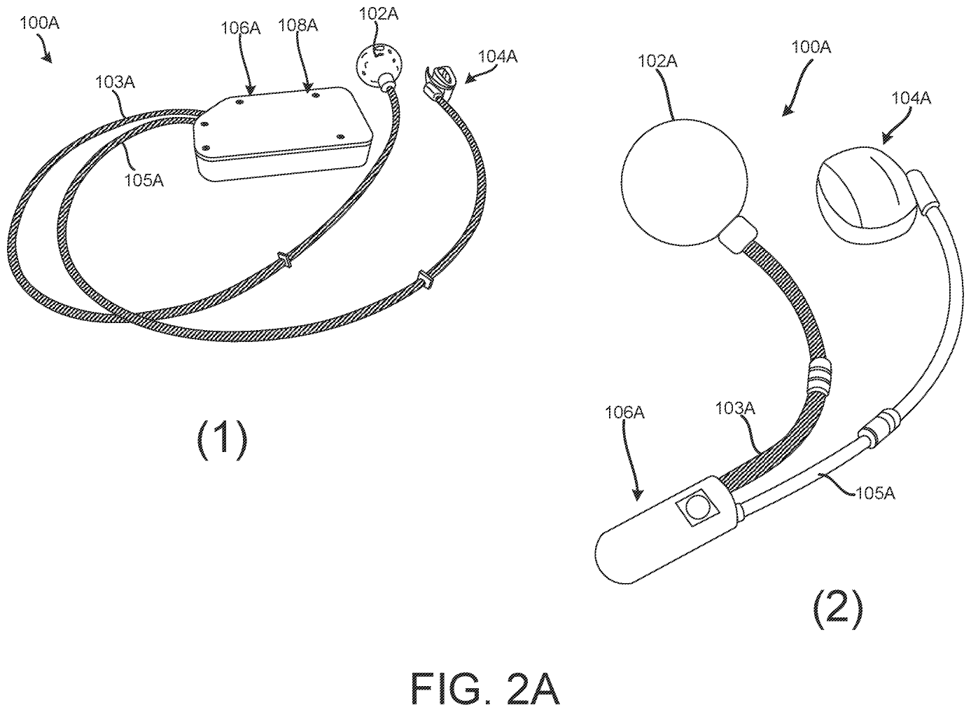

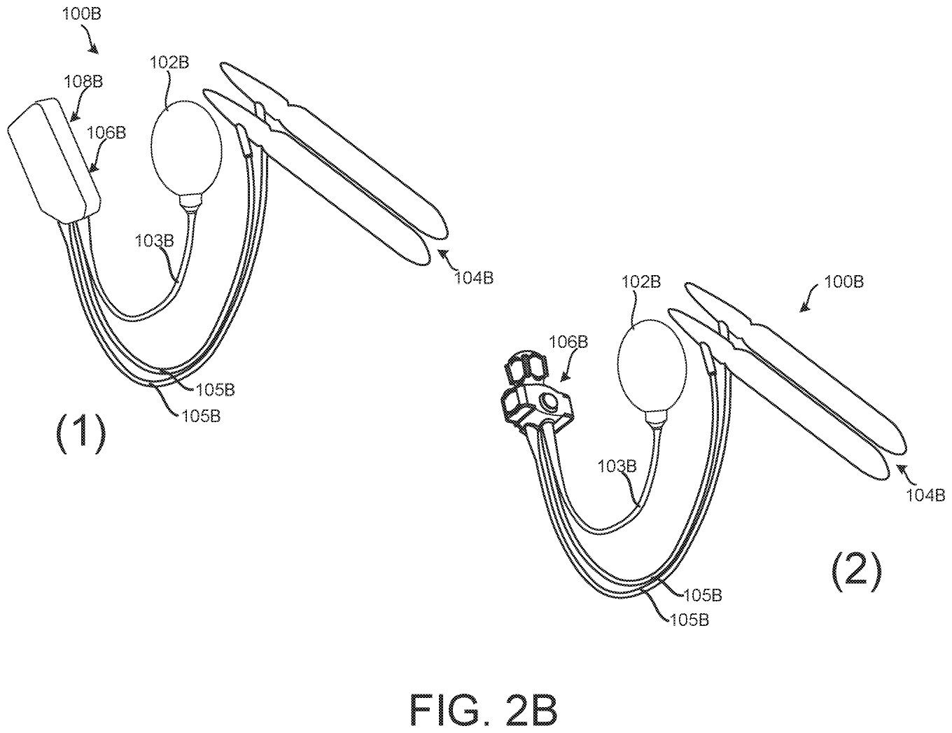

The example implantable fluid operated device 100 may be representative of a number of different types of implantable fluid operated devices. For example, the device 100 shown in FIG. 1 may be representative of an artificial urinary sphincter 100A as shown in FIG. 2A. The example artificial urinary sphincter 100A includes a pump assembly 106A. In the example shown in FIG. 2A(1), a control system 108A controls, for example, electronically controls, operation of the pump assembly 106A to provide for the transfer of fluid between a reservoir 102A and an inflatable cuff 104A. In the example shown in FIG. 2B, the pump assembly 106A may be manually controlled. A first conduit 103A connects the pump assembly 106A/control system 108A with the reservoir 102A. A second conduit 105A connects the pump assembly 106A/control system 108A with the inflatable cuff 104A. In some examples, the device 100 shown in FIG. 1 may be representative of an inflatable penile prosthesis 100B as shown in FIG. 2B. The example penile prosthesis 100B includes a pump assembly 106B. In the example shown in FIG. 2B(1), a control system 108B controls, for example, electronically controls, operation of the pump assembly 106A to provide for the transfer of fluid between a fluid reservoir 102B and inflatable cylinders 104B. In the example shown in FIG. 2B(2), the pump assembly 106B may be manually controlled. A first conduit 103B connects the pump assembly 106B/control system 108B with the reservoir 102B. One or more second conduits 105B connect the pump assembly 106A/control system 108A with the inflatable cylinders 104B. The principles to be described herein may be applied to these and other types of implantable fluid devices that rely on a pump assembly to provide for the transfer of fluid between the different fluid filled implant components to achieve inflation, deflation, pressurization, depressurization, deactivation and the like for effective operation. The example devices 100A, 100B may include electronic control systems 108A, 108B to provide for the monitoring and control of pressure and/or fluid flow through the respective devices 100A, 100B. The principles to be described herein may also be applied to implantable fluid operated devices that are manually controlled.

As noted above with respect to FIG. 1, the pump assembly can include one or more pumps and one or more valves positioned within a fluid circuit of the pump assembly to control the transfer fluid between the fluid reservoir and the inflatable member. In some examples, the pump(s) and/or the valve(s) are electronically controlled. In some examples, the pump(s) and/or the valve(s) are manually controlled. In some examples, the pump assembly includes a fluid manifold having fluidic channels formed therein, defining the fluid circuit. In an example in which the pump assembly is electronically powered and/or controlled, the manifold may be a hermetic manifold that can contain and segment the flow of fluid from electronic components of the pump assembly, to prevent leakage and/or gas exchange. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, activation and deactivation of the components of the implantable fluid operated device to provide for patient safety and device efficacy.

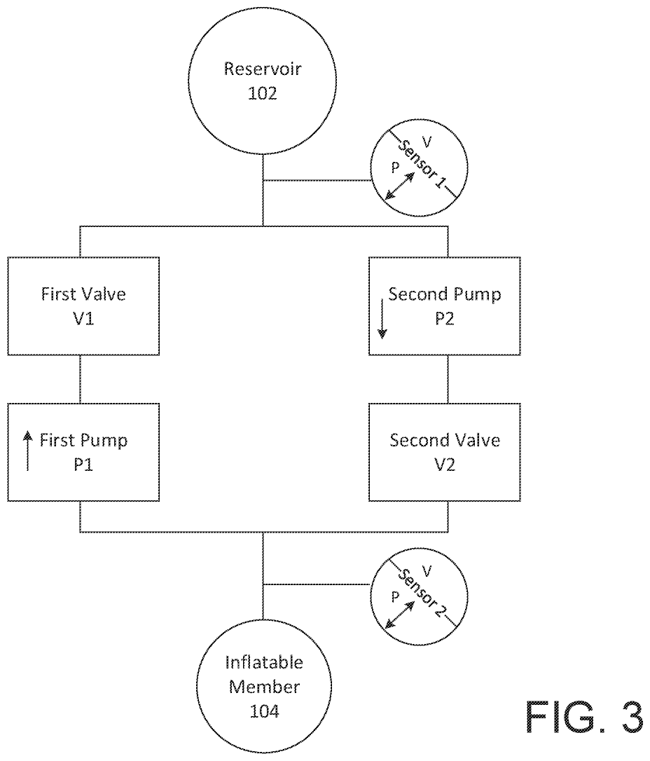

FIG. 3 is a schematic diagram of an example fluidic architecture for an implantable fluid operated device, according to an aspect. The schematic diagram shown in FIG. 3 is just one example arrangement. The fluidic architecture of an implantable fluid operated device can include other orientations of fluidic channels, valve(s), pressure sensor(s) and other components. A fluidic architecture that can accommodate back pressure, pressure surges and the like enhances the performance, efficacy and efficiency of the fluid operated device 100.

The example fluidic architecture shown in FIG. 3 includes channels guiding the flow of fluid between the reservoir 102 and the inflatable member 104. In the example shown in FIG. 3, a first valve V1 in a first fluidic channel controls the flow of fluid, generated by a first pumping device P1, from the inflatable member 104 to the reservoir 102. A second valve V2 in a second fluidic channel controls the flow of fluid, generated by a second pumping device P2, from the reservoir 102 to the inflatable member 104. In the example shown in FIG. 3, a first pressure sensing device S1 senses a fluid pressure at the reservoir 102, and a second pressure sensing device S2 senses a fluid pressure at the inflatable member 104. The first and second pressure sensing devices S1, S2 may provide for the monitoring of fluid flow and/or fluid pressure in the fluidic channels. In the arrangement shown in FIG. 3A, one of the first pump P1 or the second pump P2 is active, while the other of the first pump P1 or the second pump P2 is in a standby mode, such that the first and second pumps do not typically operate simultaneously. For example, operation of the first pump P1 (with the second pump P2 in the standby mode) may provide for the deflation of the inflatable member 104, and operation of the second pump P2 (with the first pump P1 in the standby mode) may provide for the inflation of the inflatable member 104. The valves V1, V2 may provide for the selective sealing of the respective fluidic channel(s) so as to maintain a set state of the fluid operated device. In some implementations, the valves V1, V2 may facilitate the transition between states (i.e., inflated and deflated states) of the fluid operated device. For example, selective sealing of the respective fluidic channel(s) by the valves V1, V2 may maintain an inflated state or a deflated state of the inflatable member 104. Interaction with the valves V1, V2 (and the corresponding change in fluid flow through the fluidic architecture of the device) may change the set state of the fluid operated device. Valves V1, V2 that maintain the set state of the device until the patient requires a change in the set state of the device and initiates the required change in the set state of the device provide enhanced patient safety and improved device efficacy.

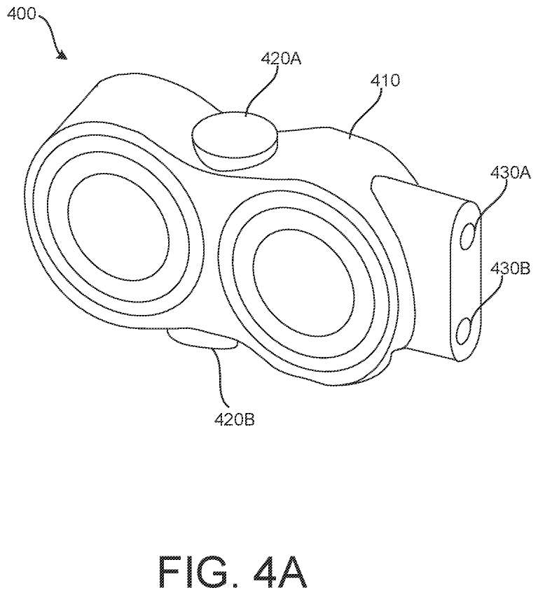

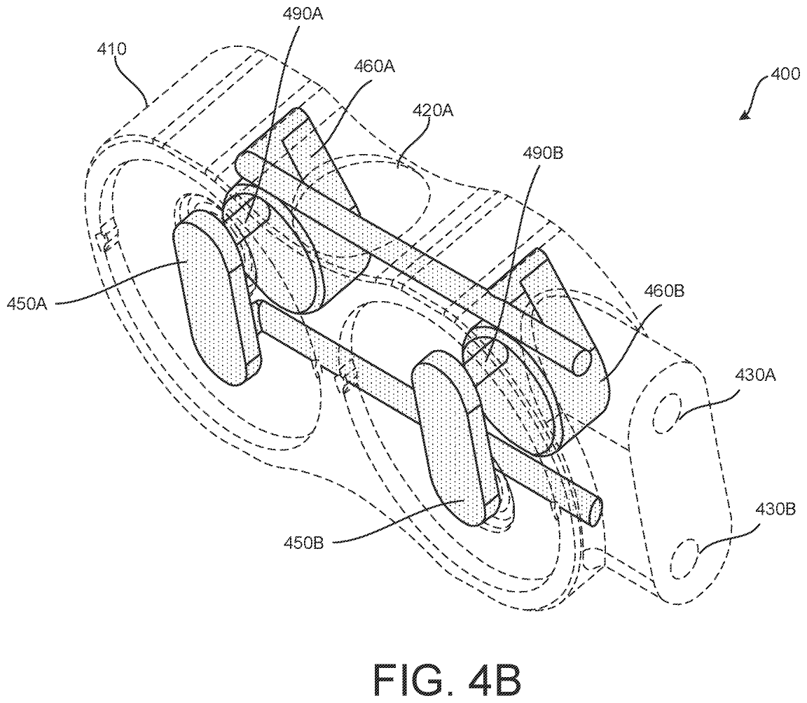

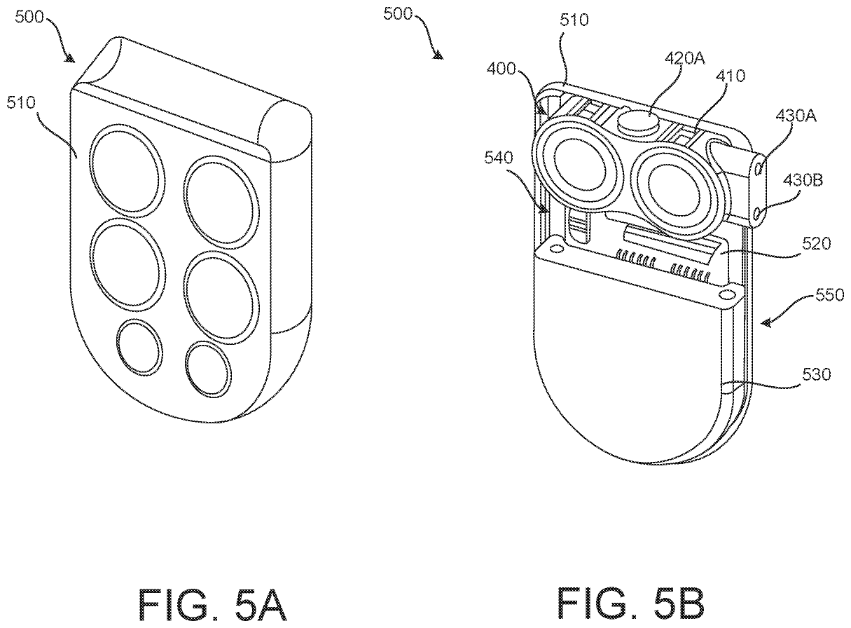

FIGS. 4A and 4B are perspective views of an example manifold 400 for use with a pumping assembly of an implantable fluid operated device. In FIG. 4B, a housing 410 of the example manifold 400 is transparent, so that an arrangement of internal fluidics components (valve(s), pump(s), sensor(s) and the like) of the manifold 400 is visible. FIGS. 5A and 5B are perspective views of an example pump assembly 500 including the manifold 400 and an electronic control system 550. In FIG. 5B, a portion of a housing 510 of the pump assembly 500 has been removed so that internal components of the pump assembly 500 are visible.

The example manifold 400 may employ a fluidic architecture such as the fluidic circuit defined by the schematic diagram shown in FIG. 3, or other fluidic architecture. The fluidic architecture of the manifold 400 may provide for the controlled transfer and monitoring of fluid in an implantable fluid operated device (such as the example devices 100 illustrated in FIGS. 2A and 2B), between the fluid reservoir 102 and the inflatable member 104.

The manifold 400 may include a housing 410. Fluid passageways may be defined within the housing 410, with fluidics components positioned within the fluid passageways. In some examples, the housing 410 may be manufactured from a solid piece of material. In some examples, the housing 410 may be molded, for example, injection molded. In some examples, the housing 410 is made of a metal material such as, for example, titanium, steel, or other biocompatible material. This may allow fluidics components to be installed in fluid passageways defined within the housing 410, and the fluid passageways to be sealed. The manifold 400/housing 410 manufactured in this manner may be hermetic, such that fluids flowing through the manifold 400 and components received in the manifold 400 are contained within the manifold 400. In a situation in which one or more of the fluidics components includes a non-biocompatible material, the hermetic nature of the manifold 400 may prevent leaching of these materials into the body of the patient, thus improving patient safety considerations.

In the example arrangement shown in FIG. 4B, the manifold 400 includes a first pump 450A in fluidic communication with a first valve 460A via a first fluid passageway 490A, and a second pump 450B in fluidic communication with a second valve 460B via a second fluid passageway 490B. The first pump 450A and the first valve 460A may direct fluid out of the manifold 400 through a first outlet port 430A to the reservoir 102 of the fluid operated device 100. The second pump 450B and the second valve 460B may direct fluid out of the manifold 400 through a second outlet port 430B to the inflatable member 104 of the fluid operated device 100. A first pressure sensing device 420A senses a fluid pressure of fluid flowing between the manifold 400 and the reservoir 102. A second pressure sensing device 420B senses a fluid pressure of fluid flowing between the manifold 400 and the inflatable member 104.

In some examples, the first valve 460A and/or the second valve 460B are normally open valves. In an arrangement in which the first and second valves 460A, 460B are normally open valves, the second valve 460B may be actuated to cause the second valve 460B to close while the first pump 450A operates to cause fluid to flow from the manifold 400 to the reservoir 102. Similarly, the first valve 460A may be actuated to cause the first valve 460A to close while the second pump 450B operates to cause fluid to flow from the manifold 400 to the inflatable member 104. Normally open valves may enhance patient safety considerations, for example, providing for the relief of pressure at the inflatable member 104 in the event of faults, failures, blockages and the like within the fluidic s architecture.

As discussed above, in some examples, control system components are incorporated into the pump assembly 500, to control and monitor operation of the pump assembly 500, and/or to provide for communication with external device(s). For example, as shown in FIGS. 5A and 5B, an electronic control system 550 may be incorporated into the pump assembly 500, together with the fluidics architecture and components in the manifold 400. FIG. 5A illustrates a stacked arrangement of components in the manifold 400. FIG. 5B illustrates a vertical arrangement of components in the manifold 400. The electronic control system 550 may include, for example, a printed circuit board (PCB) 520, a power storage device 530, battery 530, and other such electronic components. In some examples, the PCB 520 may include a processor providing processing capability, a memory, a communications module providing for communication with other electronic components, sensors and the like, as well as communication with external devices, control functionality providing for control of operation of the device, and the like. In some examples, the PCB 520 provides for the processing of inputs such as pressure and/or fluid flow measurements received from sensors of the device, the application of control algorithms to the received inputs, and the output of control functionality based on the application of the algorithms. The electronic components may be received in an electronics compartment 540 of the pump assembly 500. The electronic components may control operation of the fluidic components received in the fluid passageways in the manifold 400 as described above, may monitor fluid flow volume, fluid pressure and the like at various sections of the flow through the manifold 400 based on information received from the first and second pressure sensing devices 420A, 420B, may communicate with external devices to provide for user control and monitoring of the fluid operated device, and the like. In this type of arrangement, the hermetic manifold 400/housing 410 may isolate fluids flowing through the manifold 400 from electronic components received in the electronics compartment 540. The hermetic nature of the manifold 400 may prevent fluid leakage into the electronics compartment, and may prevent gas exchange between the manifold 400 and the electronics compartment, thus improving reliability, durability and functionality of the device, and further improving patient safety considerations.

As noted above, one or more pressure sensors may be included in the pump assembly for an implantable fluid activated device such as, for example, the devices 100 described above with respect to FIGS. 2A and 2B. In the case of electronically controlled devices, one or more pressure sensors may enable automated regulation of a state of the inflatable member and fluid supplied thereto. The inclusion of one or more pressure sensors also improved diagnostic capabilities, particularly related to isolating fluid flow issues, leakage issues and the like in the fluidic passageways, into and out of the reservoir, into and out of the inflatable member, and the like. Identification of these types of flow related issues provide for early intervention and correction. In some examples, the inclusion of one or more pressure sensors allows for dynamic control of fluid pressure, particularly within the inflatable member, to account for fluctuations due to physical activity. In some examples, the inclusion of one or more pressure sensors provides for the monitoring and control of fluid flow rates. In some examples, pressure sensor(s) included in the pump assembly for an implantable fluid activated device such as, for example, the devices 100 described above with respect to FIGS. 2A and 2B are made of bio-compatible materials, and are relatively compact and power efficient, to provide for monitoring and control of fluid pressure and/or fluid flow through the device, to preserve patient safety with minimal impact on device size and power consumption.

In some examples, the pump assembly includes multiple pressure sensors, as in, for example, the fluidic architecture shown in FIG. 3, which includes two exemplary pressure sensors. In some examples, the pump assembly includes as few as one pressure sensor. In an example including only one pressure sensor, the pressure sensor may be positioned so as to measure pressure at or near the inflatable member. For example, the pressure sensor may be positioned so as to measure fluid pressure in the inflatable member and/or fluid pressure and/or fluid flow into and out of the inflatable member.

In some examples, an electronically controlled pump assembly may provide for measurement of pressure at one or more positions within the pump assembly through the measurement of current at the one or more positions. In some examples, this may be achieved through the placement of a piezoelectric element such as a piezoelectric diaphragm in combination with a passive check valve at the desired position. An increase or a decrease in pressure will affect the deformation of the piezoelectric element. If a deformation of the piezoelectric element (and a corresponding change in voltage) is detected while the piezoelectric pump is not activated, the change in voltage will be indicative of a pressure change, and thus the piezoelectric pump can also function as a pressure sensor.

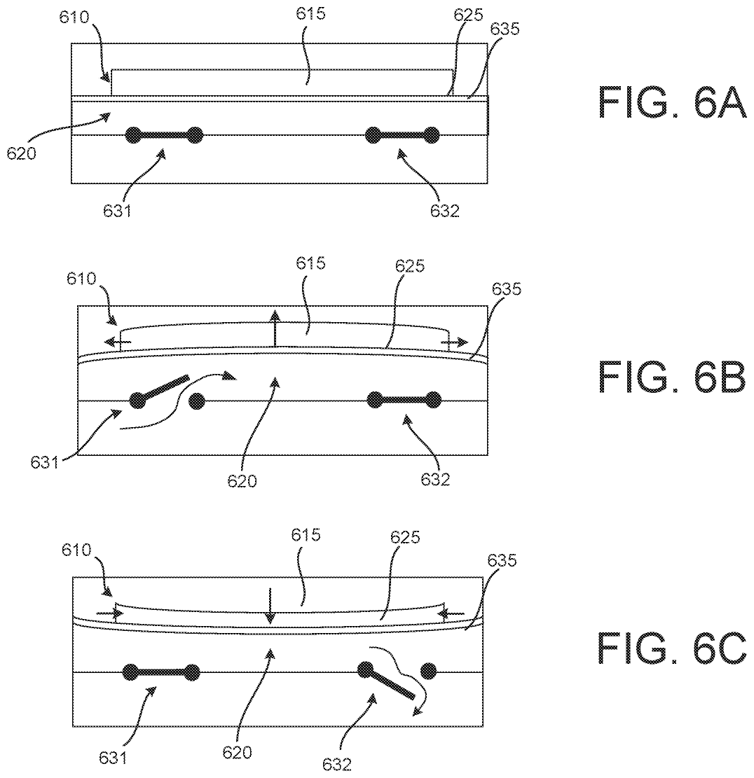

FIG. 6A illustrates a piezoelectric diaphragm 610 positioned in a fluid chamber 620 of a piezoelectric diaphragm pumping device that can provide for the pumping of fluid and also the sensing of pressure. In this example, the piezoelectric diaphragm 610 is positioned along an edge portion of the chamber 620, and includes a single layer disc 615 made of a piezoelectric material (for example, a piezo-ceramic disc) mounted on a plate 625 or membrane 625 attached to an insulative diaphragm 635. A first check valve 631 is positioned at a first side of the chamber 620, for example, an inlet end of the chamber 620, corresponding to a first end portion of the piezoelectric diaphragm 610, regulating flow through the chamber 620 in a first direction. A second check valve 632 is positioned at a second side of the chamber 620, for example, an outlet end of the chamber 620, corresponding to a second end portion of the piezoelectric diaphragm 610, regulating flow through the chamber 620 in a second direction. Application of a voltage, or an increase in voltage, causes deformation of the piezo-ceramic disc 615 and a corresponding upstroke of the membrane 625 and diaphragm 635, as shown in FIG. 6B. This upstroke of the disc 615 corresponding to a supply stroke draws fluid into the chamber 620 through the first check valve 631 to fill the chamber 620. Release of the voltage, or a decrease in voltage, causes deformation of the disc 615 and a corresponding down stroke, as shown in FIG. 6C. This down stroke of the disc 615 corresponding to a pressure stroke displaces fluid out of, or expels fluid from the chamber 620 through the second check valve 632. This pumping cycle can be repeated to continue to pump fluid into and out of, or through, the chamber 620.

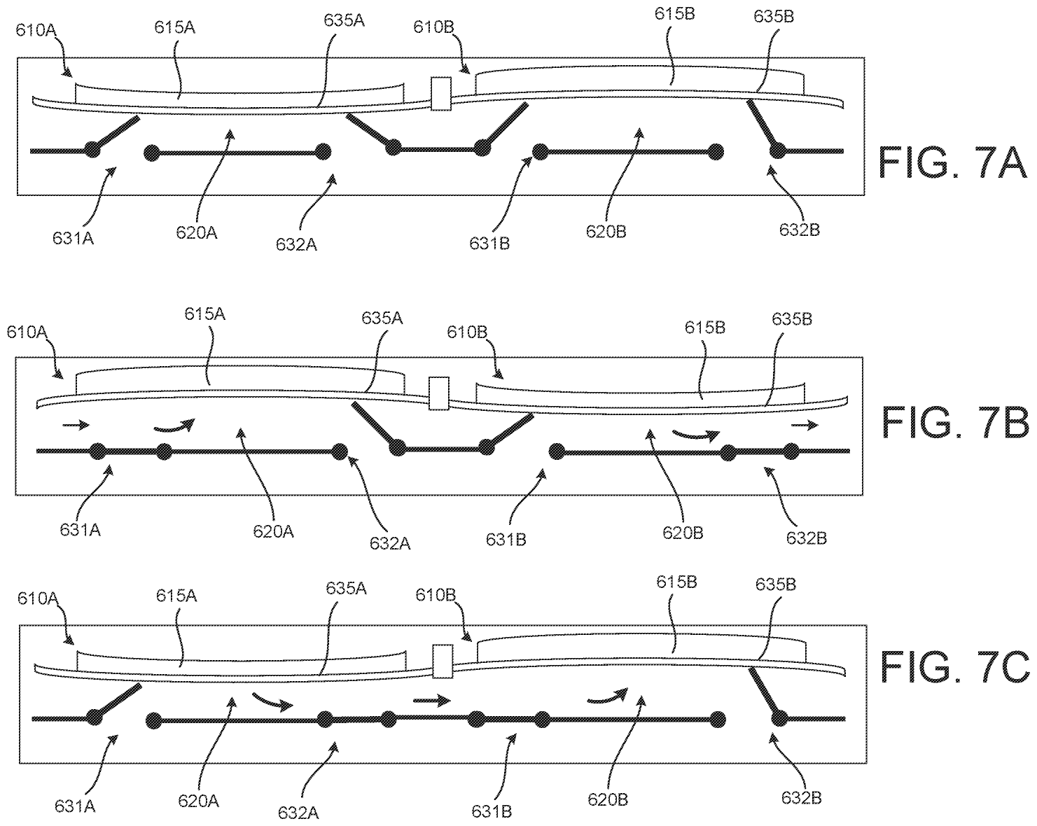

FIGS. 7A-7C schematically illustrate operation of a dual piezoelectric pump and valve manifold device. In particular, FIGS. 7A-7C illustrate operation of a dual piezoelectric pump and valve device through first, second and third phases of a pumping cycle of fluid through the dual piezoelectric pump and valve device.

In the first phase shown in FIG. 7A, a first check valve 631A and a second check valve 632A are in a closed position such that fluid does not flow into or out of a first chamber 620A corresponding to a first piezoelectric diaphragm 610A. Similarly, a first check valve 631B and a second check valve 632B are in a closed position such that fluid does not flow into or out of a second chamber 620B corresponding to a second piezoelectric diaphragm 610B.

In response to an application of voltage, a piezo-ceramic disc 615A and membrane 635A of the first piezoelectric diaphragm 610A perform an upstroke, or supply stroke, and a piezo-ceramic disc 615B and membrane 635B of the second piezoelectric diaphragm 610B perform a downstroke, or pressure stroke, from the respective first phase positions shown in FIG. 7A to the respective second phase positions shown in FIG. 7B. Voltage may be applied to the piezo-ceramic disc 615A based on, for example, a fluid pressure and/or a fluid flow rate measured by one of the pressure sensors included in the fluidic architecture described above. Upstroke of the first piezoelectric diaphragm 610A decreases a pressure in the first chamber 620A, opening the first check valve 631A and allowing fluid to flow through the first check valve 631A and into the first chamber 620A, while the second check valve 632A remains closed. Downstroke of the second piezoelectric diaphragm 610B increases a pressure in the second chamber 620B, opening the second check valve 632B and allowing fluid to flow out of the second chamber 620B and through the second check valve 632B, while the first check valve 631B remains closed.

In response to removal of the voltage, the piezo-ceramic disc 615A and membrane 635A of the first piezoelectric diaphragm 610A perform a downstroke, or pressure stroke, and the piezo-ceramic disc 615B and membrane 635B of the second piezoelectric diaphragm 610B perform an upstroke, or supply stroke, from the respective second phase positions shown in FIG. 7B to the respective third phase positions shown in FIG. 7C. Removal of the voltage applied to the piezo-ceramic disc 615A may be based on, for example, a fluid pressure and/or a fluid flow rate measured by one of the pressure sensors included in the fluidic architecture described above. Downstroke of the first piezoelectric diaphragm 610A increases a pressure in the first chamber 620A, closing the first check valve 631A and opening the second check valve 632A, allowing fluid to flow through the second check valve 632A and into the fluid channel toward the second chamber 620B. Upstroke of the second piezoelectric diaphragm 610B decreases a pressure in the second chamber 620B, opening the first check valve 631B and allowing fluid to flow into the second chamber 620B, while the second check valve 632B remains closed.

Thus, the first, second and third phases of the pumping cycle of the dual piezoelectric pump and valve device shown in FIGS. 7A-7C illustrate the refilling of fluid in the first chamber 620A and the discharge of fluid accumulated in the second chamber 620B in going from the first phase (FIG. 7A) to the second phase (FIG. 7B), and the discharge of fluid accumulated in the first chamber 620A and the refilling of fluid into the second chamber 620B in going from the second phase (FIG. 7B) to the third phase (FIG. 7C).

In the example described above with respect to FIGS. 7A-7C, the dual piezoelectric pump and valve device includes a first check valve 631A, 631B and a second check valve 632A, 632B respectively associated with the flow through each chamber 620A, 620B. In some implementations, operation of the second check valve 632A of the first chamber 620A and the first check valve 631B of the second chamber 620B can be replaced with a single valve (not shown in FIGS. 7A-7C) that can control the flow between the first chamber 620A and the second chamber 620B in a similar manner to that which is described above with respect to FIGS. 7A-7C.

In some examples, a current-mode sensing method may be applied to determine pressure in a piezoelectric diaphragm pump. As current and pressure are linearly interrelated, pressure can be inferred from the amount of current required to move the diaphragm. In this type of current-mode sensing, pressure can be sensed at each pumping cycle as described above, based on the amount of current required to move the diaphragm and fill/empty the respective chamber.

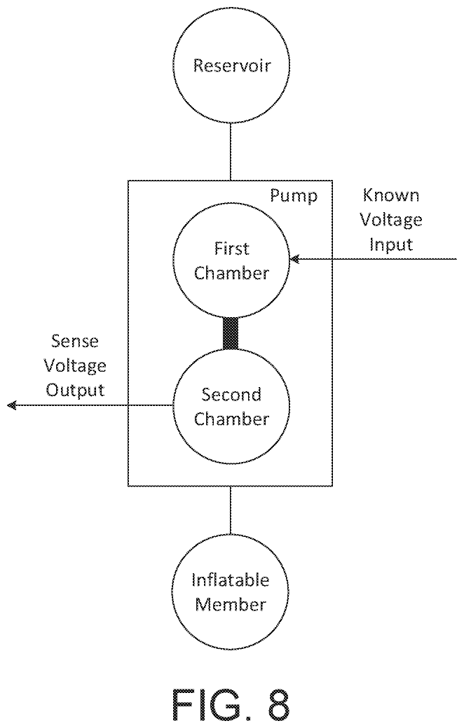

In some examples, an induced-response method may be applied to determine pressure. The induced-response method may make use of the ability of piezoelectric materials to convert movement into voltage (in addition to moving in response to the application of electrical stimulus, as described above). As the electro-mechanical actuation and responses of piezoelectric materials are associated with alternating current (AC) signals, the above-described use of the pump as a sensor (in, for example, the piezoelectric diaphragm pump as described above) can only measure changes in pressure. In some examples, this can be overcome by controlling an input to one fluid chamber, and measuring an output at another fluid chamber. FIG. 8 is a schematic diagram of an example dual piezoelectric pump manifold configuration, such as the example dual piezoelectric pump and valve device shown in FIGS. 7A-7C, having multiple chambers arranged in series. In this example arrangement, the first chamber (for example, the first chamber 620A) may be connected to the second chamber (for example, the second chamber 620B) by a fluid passageway. A known stimulus (i.e., a known voltage level, or a known pulse level) is input at the first chamber, and the output at the second chamber (a voltage level, or a pulse magnitude) is detected. In some examples, a static pressure can be determined based on a known pulse input applied to the first chamber, and the resultant pulse output measured at the second chamber.

As established above, the ability to accurately measure and monitor pressure in an implantable fluid operated device as described herein is essential for proper operation of the device and device efficacy, and to ensure patient safety. In some situations, it may be necessary to also be able to identify atmospheric pressure, and to adjust operation of the device accordingly to account for differences from a calibrated atmospheric pressure level in operation and control of the device. For example, the example devices 100 described above operate based on a principle of differential pressure. With a relatively high pressure in the reservoir 102, a relatively low pressure will be present in the inflatable member 104. Similarly, with a relatively low pressure in the reservoir 102, a relatively high pressure will be present in the inflatable member 104. If the device 100 is calibrated, for example, at sea level, variances in atmospheric pressure (i.e., above or below sea level) may affect pressure measurement and monitoring in the fluid channels of the device 100, and may affect operation of the device 100. Control of fluid pressure within the device 100, and in particular at various different positions within the device 100, may provide for monitoring of pressure within the device 100 and control of device operation independent of atmospheric pressure.

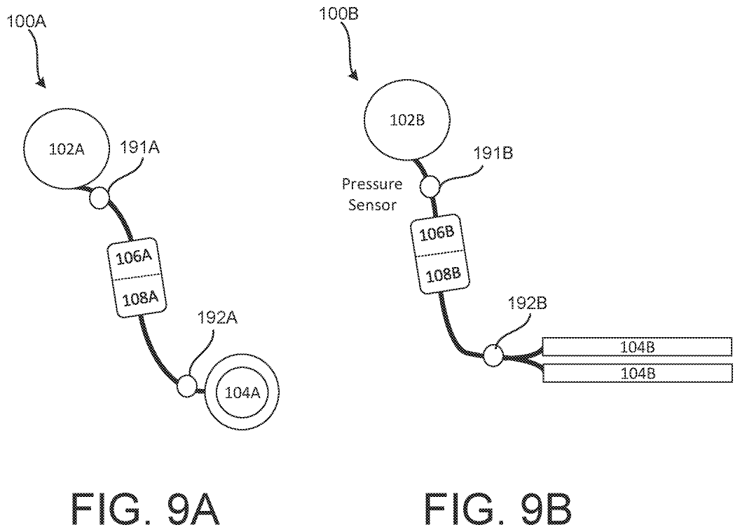

For example, absent a mechanism for accounting for atmospheric pressure changes, spikes, and the like, an increase or a decrease in atmospheric pressure (from the calibration pressure) may cause the device 100 to incorrectly pump fluid to the inflatable member 104, or back to the reservoir 102, to account for the offset in atmospheric pressure. FIGS. 9A and 9B illustrate the example devices 100 described above, in the form of the artificial urinary sphincter 100A and the example inflatable penile prosthesis 100B. Each of the example devices 100 includes inline pressure sensors. For example, a first inline pressure sensor 191 (191A, 191B) is positioned close to the reservoir 102, and a second inline pressure sensor 192 (192A, 192B) is positioned close to the inflatable member 104 of each device 100.

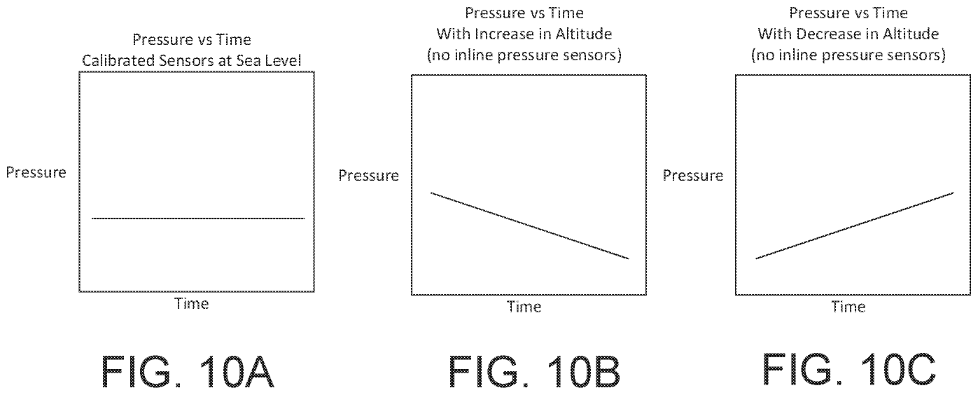

When calibrated, for example, at sea level, any pressure differential between the reservoir 102 and the inflatable member 104 is accounted for, or offset, or known, based on a pressure measurement provided by the first pressure sensor 191 and the second pressure sensor 192. When functioning properly, the first and second pressure sensors 191, 192 should experience the same decrease or increase in pressure in response to a sudden increase in altitude, or a sudden decrease in altitude, thus maintaining a substantially constant pressure level, as illustrated by the graph shown in FIG. 10A. The use of inline pressure sensors as described may allow for measurements taken by the first and second pressure sensors 191, 192 to be transmitted to the electronic control system 108, to be monitored, and in the event of an increase or decrease in pressure, internal algorithms (for example, applied or carried out by components of the control system 108 of the device 100) can use the pressure measurements to account for the difference and adapt the pumping of fluid through the device to maintain a proper inflated/deflated state of the inflatable member 104.

In particular, the graph shown in FIG. 10B illustrates that, in response to an increase in altitude, a decrease in system pressure is experienced. Without the first and second inline pressure sensors 191, 192 as described above, and a control algorithm that provides for correction of pressure levels to account for changes in altitude, the observed decrease in pressure could trigger the device 100 to (erroneously) increase pumping of fluid to the inflatable member 104. This may cause over-pressurization of the cuff 104A and damage to the urethra and/or device failure, or unintended inflation of the inflatable cylinders 104B. Similarly, the graph shown in FIG. 10C illustrates that, in response to a decrease in altitude, an increase in system pressure is experienced. Without the first and second inline pressure sensors 191, 192 as described above, and a control algorithm that provides for correction of pressure levels to account for changes in altitude, the observed increase in pressure could trigger the device 100 to (erroneously) decrease pumping of fluid to the inflatable member 104/deflate the inflatable member 104 and re-direct fluid from the inflatable member 104 back to the reservoir 102. This may result in an under-pressurization of the cuff 104A on the urethra and patient leakage, or unintended deflation of the inflatable cylinders 104B.

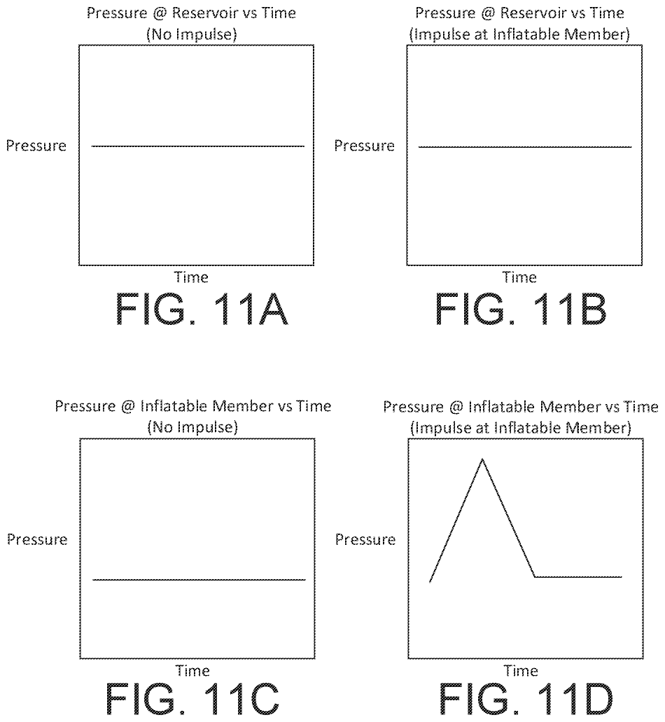

The graphs shown in FIGS. 11A-11D illustrate the effect of a single, abrupt impulse or impact experienced at the inflatable member 104 due to various physical actions such as, for example, exercise and the like which may temporarily impinge on the inflatable member 104 and cause an intermittent spike in pressure. Under normal, calibrated conditions (and in the absence of an impulse as described above), any pressure differential between the reservoir 102 and the inflatable member 104 is accounted for, or offset, or known, based on pressure measurements provided by the first and second pressure sensors 191, 192 as described above, and as shown in FIGS. 11A and 11C. Further, based on the inline placement of the first and second pressure sensors 191, 192, the system may detect that, in this scenario the sudden spike in pressure is detected only by the second pressure sensor 192 (at or near the inflatable member 104) as shown in FIG. 11D, but not by the first pressure sensor 191 (at or near the reservoir 102) as shown in FIG. 11B. The system may then take action based on an established decision algorithm to increase pumping action, decrease pumping action, or take no action. For example, if continued pressure monitoring detects that the pressure increase is not sustained over a period of time, and that pressure returns to within the expected calibrated range as shown in FIG. 11D, no action is taken. This may allow the device 100 to adapt to specific use scenarios relatively quickly, while also enhancing patient safety and comfort.

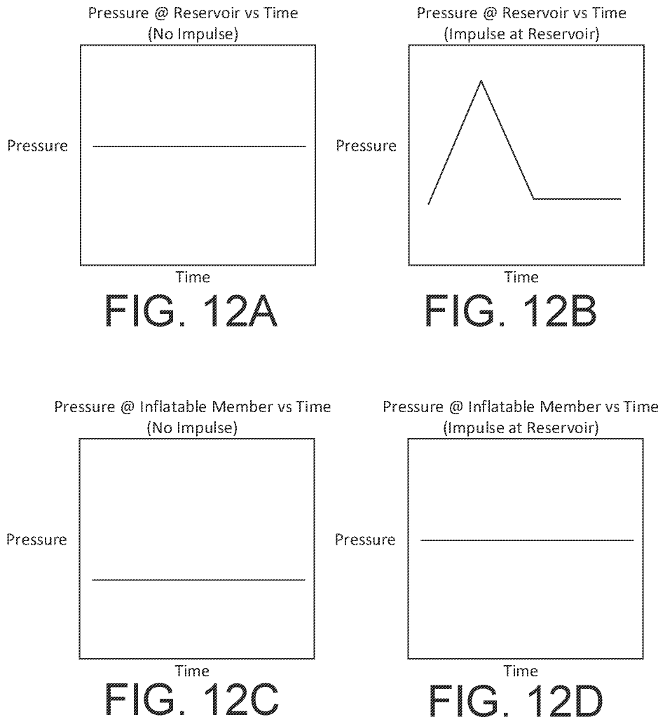

The graphs shown in FIGS. 12A-12D illustrate the effect of a single, abrupt impulse or impact experienced at the reservoir 102 due to various physical actions such as, for example, a fall and the like which may temporarily impinge on the reservoir 102 and cause an intermittent spike in pressure. Under normal, calibrated conditions (and in the absence of an impulse as described above), any pressure differential between the reservoir 102 and the inflatable member 104 is accounted for, or offset, or known, based on pressure measurements provided by the first and second pressure sensors 191, 192 as described above, and as shown in FIGS. 12A and 12C. Further, based on the inline placement of the first and second pressure sensors 191, 192, the system may detect that, in this scenario the sudden spike in pressure is detected only by the first pressure sensor 191 (at or near the reservoir 102) as shown in FIG. 12B, but not by the second pressure sensor 192 (at or near the inflatable member 104) as shown in FIG. 12D. The system may then take action based on an established decision algorithm to increase pumping action, decrease pumping action, or take no action. For example, if continued pressure monitoring detects that the pressure increase is not sustained over a period of time, and that pressure returns to within the expected calibrated range as shown in FIG. 12B, no action is taken. This may allow the device 100 to adapt to specific use scenarios relatively quickly, while also enhancing patient safety and comfort.

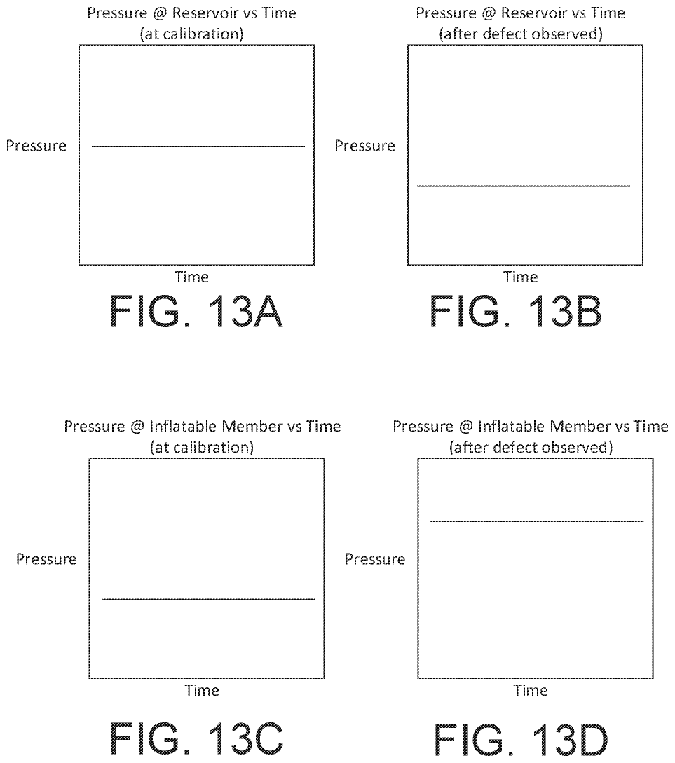

The graphs shown in FIGS. 13A-13D illustrate the effect of a relatively long term different or drift in set pressure values between the reservoir 102 and the inflatable member 104, or a time to reach the set pressure values is noticeably increased. These events may be indicative of a blockage in one of the fluid passageways of the device 100, or other type of damage or malfunction of the device 100, and may provide notification to the patient and/or physician for correction. In normal operation, an offset between pressure levels measured by the first and second inline pressure sensors 191, 192 should remain essentially constant, as shown in FIGS. 13A and 13C. A component failure, a leak, a blockage or other such disruption would generate a surge in pressure, or a decrease in pressure, based on the type of failure and the location of the failure within the device 100, as shown in FIGS. 13B and 13D. The detection of a sustained decrease or surge in pressure can provide an alert to the patient and/or to the physician to provide for correction, thus enhancing patient safety and comfort.

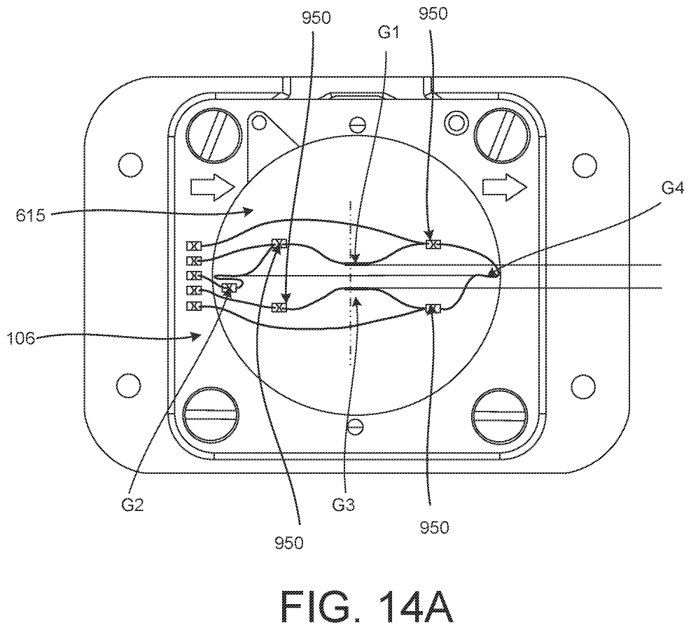

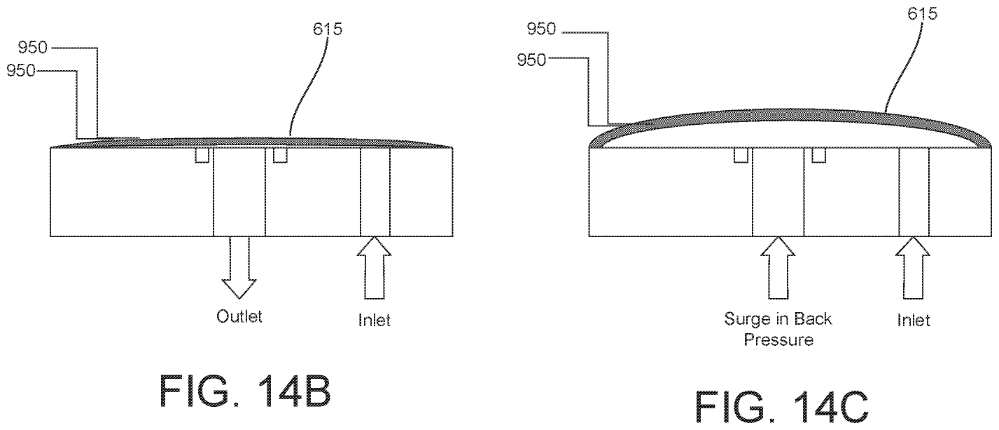

As noted above, the example inline pressure sensors 191, 192 shown in FIGS. 9A and 9B may be positioned in the fluid passageways of the implantable fluid activated device 100. In some examples, the inline pressure sensors 191, 192 can include a diaphragm positioned in the fluid passageway. For example, the first pressure sensor 191 can include a diaphragm positioned in the fluid passageway and facing the reservoir 102, and the second pressure sensor 192 can include a diaphragm positioned within the fluid passageway and facing the inflatable member 104. Deflection of the diaphragm can be detected/measured and an algorithm (for example carried out by the electronic control system 108) can covert the detected movement or deflection of the diaphragm into a pressure. In some examples, the deflection of the diaphragm may be measured by a strain gauge positioned on the diaphragm. FIG. 14A illustrates one example of strain gauges 950 mounted on the diaphragm within a fluid passageway of the pump assembly 106. In some examples, the diaphragm is made of a bio-compatible material such as, for example, Titanium. In some examples, the diaphragm is coated in an elastic material such as, for example, a silicone material, a ceramic material and the like, that provides a moisture barrier on the diaphragm while also allowing for the transfer of a signal from the strain gauge. In some examples, the device 100 can communicate with an external device (for example, through a communication module of the electronic control system 108). Communication with the external device can provide for the exchange of information such as, for example, atmospheric pressure readings (that allow the internal device 100 to adjust pressures as necessary), internal pressure measurements, alerts and the like.

As described above, the ability to detect other than normal pressure level(s) in the device 100, and to adapt the operation of the device 100 in response to detection of the other than normal pressure level(s) enhances patient safety and device efficacy. For example, as described above with respect to FIGS. 11A-11D, a detected spike or increase in pressure may cause the device 100 to adjust pumping action. In some situations, the decision to adjust pumping action may be based on an observed duration of the increased pressure. For example, in the case of the artificial urinary sphincter 100A, the insertion of a catheter can cause a relatively rapid increase in pressure, particularly if the cuff 104A has not been deflated prior to insertion of the catheter. For example, in some situations, the patient may be incapacitated and/or unable to communicate the presence of the implanted artificial urinary sphincter. Insertion of the catheter with the cuff 104A in the inflated condition causes a rapid buildup of pressure in the device 100A, that is sustained and/or continues to increase as the catheter is inserted. In this example, the detection of this type of sustained pressure spike may cause the electronic control system 108A to actuate the pump assembly 106A to deflate the cuff 104A, thus opening the cuff 104A and allowing the catheter to be inserted without damaging the cuff 104A and/or the urethra.

In some examples, the spike in pressure is detected by a pressure sensor within the fluid passageways of the device, including, for example, a piezoelectric element as described above, a pressure transducer, and the like. In some examples, the spike in pressure is detected based on dynamic pressure changes in a piezoelectric element. As described above, diaphragms placed positioned in the fluid passageway facing the reservoir 102A and facing the cuff 104A are deflected as fluid pressure changes. A normal state and a deflected state of the example diaphragm 615 is shown in FIGS. 14B and 14C. The dynamic pressure in response to insertion of a catheter as described above generates a voltage change that is measurable by the strain gauge(s) 950. The voltage change is indicative of a change in pressure caused by the insertion of the catheter. The electronic control system 108 can process the detected change in pressure and control the pump assembly 106 to provide for deflation/opening of the cuff 104A.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable fluid operated device, comprising:
a fluid reservoir;
an inflatable member;
a pump assembly configured to transfer fluid between the fluid reservoir and the inflatable member, including:
a manifold, including:
a housing;
at least one valve and at least one pump positioned in a fluid passageway formed in the housing;
a first fluid port in fluidic communication with the fluid reservoir; and
a second fluid port in fluidic communication with the inflatable member;
an electronic control system controlling operation of the pump assembly; and
at least one pressure sensing device in communication with the electronic control system.

2. The implantable fluid operated inflatable device of claim 1, wherein the at least one valve and the at least one pump includes:
a first pump and a first valve positioned in a first fluid passageway and in fluidic communication with the first fluid port; and
a second pump and a second valve positioned in a second fluid passageway and in fluidic communication with the second fluid port.

3. The implantable fluid operated inflatable device of claim 2, wherein the at least one pressure sensing device includes:
a first pressure sensing device positioned in the first fluid passageway and configured to measure a pressure of fluid flowing through the first fluid port and to transmit the measured pressure to the electronic control system; and
a second pressure sensing device positioned in the second fluid passageway and configured to measure a pressure of fluid flowing through the second fluid port and to transmit the measured pressure to the electronic control system.

4. The implantable fluid operated inflatable device of claim 1, wherein the at least one valve and the at least one pump forms a dual piezoelectric pump and valve manifold, including:
a first piezoelectric pump;
a second piezoelectric pump; and
a fluid channel providing for fluidic communication between the first piezoelectric pump and the second piezoelectric pump.

5. The implantable fluid operated inflatable device of claim 4, wherein
wherein
the first piezoelectric pump includes:
a first chamber;
a first piezoelectric diaphragm positioned along an edge portion of the first chamber and configured to have a voltage selectively applied thereto in response to a fluid pressure detected by at least one of the first pressure sensing device or the second pressure sensing device;
a first check valve at an inlet end of the first chamber; and
a second check valve at an outlet end of the first chamber, the second check valve of the first piezoelectric pump selectively providing fluidic communication between the first chamber and the fluid channel; and
the second piezoelectric pump includes:
a second chamber;
a second piezoelectric diaphragm positioned along an edge portion of the second chamber and configured to have a voltage selectively applied thereto in response to a fluid pressure detected by at least one of the first pressure sensing device or the second pressure sensing device;
a first check valve at an inlet end of the second chamber, the first check valve of the second piezoelectric pump selectively providing fluidic communication between the fluid channel and the second chamber; and
a second check valve at an outlet end of the second chamber.

6. The implantable fluid operated inflatable device of claim 5, wherein a pumping cycle of the dual piezoelectric pump includes:
a first phase including a supply stroke of the first piezoelectric diaphragm in coordination with a pressure stroke of the second piezoelectric diaphragm; and
a second phase including a pressure stroke of the first piezoelectric diaphragm in coordination with a supply stroke of the second piezoelectric diaphragm.

7. The implantable fluid operated inflatable device of claim 6, wherein
in the first phase, fluid is drawn into the first chamber through the first check valve of the first piezoelectric pump, and fluid is expelled from the second chamber through the second check valve of the second piezoelectric pump; and
in the second phase, fluid is expelled from the first chamber and into the fluid channel through the second check valve of the first piezoelectric pump, and fluid is drawn from the fluid channel into the second chamber through the first check valve of the second piezoelectric pump.

8. The implantable fluid operated inflatable device of claim 1, wherein the housing of the manifold is made of an injection molded metal material, with the at least one pump and the at least one valve positioned in a sealed fluid passageway defined in the injection molded metal material, such that the manifold is a hermetic manifold.

9. The implantable fluid operated inflatable device of claim 1, wherein the pump assembly includes a pump assembly housing, and wherein the manifold and the electronic control system are received in the pump assembly housing.

10. The implantable fluid operated inflatable device of claim 9, wherein the manifold is a hermetic manifold, such that components of the electronic control system within the pump assembly housing are isolated from fluid flowing through the hermetic manifold.

11. The implantable fluid operated inflatable device of claim 1, wherein the at least one pressure sensing device includes:
a first pressure sensing device positioned proximate a fluid port of the reservoir; and
a second pressure sensing device positioned proximate a fluid port of the inflatable member.

12. The implantable fluid operated inflatable device of claim 11, wherein
the first pressure sensing device includes:
a first diaphragm positioned in a fluid passageway proximate the reservoir, facing the reservoir; and
at least one first strain gauge mounted on the first diaphragm, the at least one first strain gauge being configured to measure a deflection of the first diaphragm and to transmit the measured deflection to the electronic control system; and
the second pressure sensing device includes:
a second diaphragm positioned in a fluid passageway proximate the fluid port of the inflatable member, facing the inflatable member; and
at least one second strain gauge mounted on the second diaphragm, the at least one second strain gauge being configured to measure a deflection of the second diaphragm and to transmit the measured deflection to the electronic control system.

13. The implantable fluid operated inflatable device of claim 1, wherein the at least one sensing device includes at least one piezoelectric element positioned in a fluid passageway of the implantable fluid operated device and configured to sense a fluid pressure level in the fluid passageway based on an input voltage level applied to the piezoelectric element and an output voltage level measured at the piezoelectric element.

14. The implantable fluid operated inflatable device of claim 1, wherein the electronic control system includes a printed circuit board including a processor configured to:
receive pressure level measurements from the at least one sensing device;
apply a control algorithm based on the received pressure level measurements; and
control operation of the at least one valve and the at least one pump in accordance with the applied control algorithm.

15. The implantable fluid operated inflatable device of claim 1, wherein the implantable fluid operated device is an artificial urinary sphincter or an inflatable penile prosthesis.

16. An implantable fluid operated inflatable device, comprising:
a fluid reservoir;
an inflatable member;
a pump assembly received in a housing and configured to transfer fluid between the fluid reservoir and the inflatable member, including:
a manifold;
a pump and valve device received in the manifold; and
an electronic control system configured to control operation of the pump and valve device.

17. The implantable fluid operated inflatable device of claim 16, wherein the manifold is a hermetic manifold, and the electronic control system includes a first portion received in an electronics compartment of the housing, isolated from fluid flowing through the manifold.

18. The implantable fluid operated inflatable device of claim 17, wherein the electronic control system includes a second portion that is external to the implantable fluid operated inflatable device, and is configured to communicate with of the first portion of the electronic control system, wherein the second portion is configured to receive user inputs, and to output information to the user.

19. The implantable fluid operated inflatable device of claim 16, wherein the pump and valve device is a dual piezoelectric pump and valve device, including a first piezoelectric pump in fluidic communication with a second piezoelectric pump via a fluid channel,
the first piezoelectric pump, including:
a first chamber;
a first piezoelectric element and diaphragm positioned along an edge portion of the first chamber;
a first check valve at an inlet end of the first chamber; and
a second check valve at an outlet end of the first chamber, the second check valve of the first piezoelectric pump selectively providing fluidic communication between the first chamber and the fluid channel; and
the second piezoelectric pump including:
a second chamber;
a second piezoelectric element and diaphragm positioned along an edge portion of the second chamber;
a first check valve at an inlet end of the second chamber, the first check valve of the second piezoelectric pump selectively providing fluidic communication between the fluid channel and the second chamber; and
a second check valve at an outlet end of the second chamber.

20. The implantable fluid operated inflatable device of claim 19, wherein
in an inflation mode, the electronic control system is configured to alternately apply a voltage input to the first piezoelectric element and the second piezoelectric element to cause fluid to flow through the dual piezoelectric pump in a first direction, from the fluid reservoir toward the inflatable member; and
in a deflation mode, the electronic control system is configured to alternately apply a voltage input to the first piezoelectric element and the second piezoelectric element to cause fluid to flow through the dual piezoelectric pump in a second direction, from the inflatable member toward the reservoir.
